(19) United States
(12) Patent Application Publication
(10) Pub. No.: US 2014/0371132 A1
(43) Pub. Date: Dec. 18, 2014

(54) CODON SIGNATURE FOR NEUROMYELITIS OPTICA

(71) Applicant: The Board of Regents of the University of Texas System, Austin, TX (US)

(72) Inventor: Nancy Monson, Ovilla, TX (US)

(73) Assignee: The Board of Regents of the University of Texas System, Austin, TX (US)

(21) Appl. No.: 14/352,473

(22) PCT Filed: Oct. 18, 2012
(86) PCT No.: PCT/US12/60758

§ 371 Date: Apr. 17, 2014

Related U.S. Application Data
(60) Provisional application No. 61/550,158, filed on Oct. 21, 2011.

(51) Int. Cl. C12Q 1/68; G01N 33/53

(52) U.S. Cl. CPC C12Q 1/6869; G01N 33/53; G01N 2800/285
USPC 514/1.1; 435/6.12; 436/501; 435/6.11

(57) ABSTRACT

The present invention provides for the diagnosis and prediction of neuromyelitis optica (NMO) in subject utilizing a unique a codon signature in B cells that has now been associated with NMO and not with any other autoimmune disease. More particularly, the method may comprise the steps of (a) providing a 10 B-cell containing sample from a subject, or DNA or RNA isolated therefrom; (b) determining the VH1 and/or VH4 structure of VH1NH4-expressing B-cells from said subject, (c) determining the mutational frequency VH1 and/or VH4 genes; (d) identifying the presence or absence of a codon signature associated with NMO or risk of NMO; and (e) selecting patients exhibiting said codon signature.

The present application claims benefit of priority to U.S. Provisional Application Ser. No. 61/550,158, filed Oct. 21, 2011, the entire contents of which are hereby incorporated by reference.

### BACKGROUND OF THE INVENTION

1. Field of the Invention

The present invention relates to fields of pathology, immunology and molecular biology. More particularly, the present invention relates to a pattern of somatic hypermutation or “signature” in the antibody genes of B cells that predicts and diagnoses neuromyelitis optica (NMO).

2. Description of Related Art

Neuromyelitis optica (NMO), also known as Devic's disease or Devic's syndrome, is an autoimmune, inflammatory disorder that attacks the optic nerves and spinal cord. This produces an inflammation of the optic nerve (optic neuritis) and the spinal cord (myelitis). Although inflammation may also affect the brain, the lesions are different from those observed in the related condition multiple sclerosis (MS). Spinal cord lesions lead to varying degrees of weakness or paralysis in the legs or arms, loss of sensation (including blindness), and/or bladder and bowel dysfunction. NMO is a rare disorder, which resembles MS in several ways, but requires a different course of treatment for optimal results. A likely target of the autoimmune attack at least in some patients with NMO is a protein of the nervous system cells called aquaporin 4.

NMO is similar to MS in that the body's immune system attacks the myelin surrounding nerve cells. Unlike standard MS, the attacks are not believed to be mediated by the immune system's T cells but rather by antibodies called NMO-IgG, or simply NMO antibodies. These antibodies target a protein called aquaporin 4 in the cell membranes of astrocytes, which acts as a channel for the transport of water across the cell membrane. Aquaporin 4 is found in the processes of the astrocytes that surround the blood-brain barrier, a system responsible for preventing substances in the blood from crossing into the brain. The blood-brain barrier is weakened in NMO, but it is currently unknown how the NMO-IgG immune response leads to demyelination.

### SUMMARY OF THE INVENTION

This method identifies a human subject as having or at risk of developing neuromyelitis optica (NMO) and is comprised of assessing the VH1 and/or VH4 sequences of VH1 and/or VH4-expressing B-cells from said subject, wherein the presence of one or more mutations in codons associated with NMO identifies said subject as having or at risk of developing NMO. More particularly, the method may comprise the steps of (a) providing a B-cell containing sample from a subject, or DNA or RNA isolated therefrom; (b) determining the VH1 and/or VH4 structure of VH1/VH4-expressing B-cells from said subject, (c) determining the mutational frequency VH1 and/or VH4 genes; (d) identifying the presence or absence of a codon signature associated with NMO or risk of NMO; and (e) selecting patients exhibiting said codon signature. The sample may be blood, serum, ocular fluid or tears.

The NMO codon signature may comprise a mutation in VH1 at codon 47, 54, 70, 79, 84 and/or 91, or in 2, 3, 4, 5 or all 6 of said VH1 codons. The NMO codon signature may comprise a mutation in VH4 at codon 36, 39, 45, 46, 50, 59, 61, 65, 67, 70, 86 and/or 90, or 2, 3, 4, 5, 6, 7, 8, 9, 10, 11 or all 12 of said VH4 codons. The NMO codon signature may comprise a mutation in codons 47, 54, 70, 79, 84 and/or 91 of VH1 and codons 36, 39, 45, 46, 50, 59, 61, 65, 67, 70, 86 and/or 90 of VH4, or in 2, 3, 4, 5, 6, 7, 8, 9, 10, 11, 12, 13, 14, 15, 16, 17 or all 18 of said codons. The method may further comprise assessing one or more traditional NMO risk factors. Assessing may comprise sequencing and/or PCR. The B-cells may be obtained from cerebrospinal fluid (CSF) or peripheral blood. The method may further comprise assessing J chain usage, J chain length and/or CDR3 length. The method may further comprise making a treatment decision based on the presence of said codon signature.

In another embodiment, there is provided a method of screening for an agent useful in treating neuromyelitis optica (NMO) comprising (a) providing an antibody produced by VH1 and/or VH4-expressing B-cells, said antibody genes comprising mutations at two or more codons selected from the group consisting of codons 47, 54, 70, 79, 84 and of VH1 and codons 36, 39, 45, 46, 50, 59, 61, 65, 67, 70, 86 and 90 of VH4; (b) contacting said antibodies with candidate ligand(s); and (c) assessing binding of said candidate ligand(s) to said antibodies, wherein binding of said candidate ligand(s) to said antibodies identifies said candidate ligand(s) as useful in treating NMO. The candidate ligand(s) may be a peptide or a peptoid. The NMO codon signature may comprise a least one or multiple mutations in both VH1 and VH4, such as a NMO codon signature that comprises mutations at 2, 3, 4, 5, 6, 7, 8, 9, 10, 11, 12, 13, 14, 15, 16, 17 or all 18 of said codons.

In yet another embodiment, there is provided a method of treating a subject having or at risk of developing neuromyelitis optica (NMO) comprising administering to said subject a ligand that binds to either a VH1 or VH4 antibody comprising mutations at two or more codons selected from the group consisting of codons 47, 54, 70, 79, 84 and 91 of VH1 and codons 36, 39, 45, 46, 50, 59, 61, 65, 67, 70, 86 and 90 of VH4. The ligand may be a peptide or a peptoid. The ligand may be linked to a toxin or B-cell antagonist. The NMO codon signature may comprise a least one or multiple mutations in VH1 and/or VH4 antibody genes, such as a NMO codon signature that comprises mutations at 2, 3, 4, 5, 6, 7, 8, 9, 10, 11, 12, 13, 14, 15, 16, 17 or all 18 of said codons.

It is contemplated that any method or composition described herein can be implemented with respect to any other method or composition described herein.

The use of the word “a” or “an” when used in conjunction with the term “comprising” in the claims and/or the specification may mean “one,” but it is also consistent with the meaning of “one or more,” “at least one,” and “one or more than one.”

These, and other, embodiments of the invention will be better appreciated and understood when considered in conjunction with the following description and the accompanying drawings. It should be understood, however, that the following description, while indicating various embodiments of the invention and numerous specific details thereof, is given by way of illustration and not of limitation. Many substitutions, modifications, additions and/or rearrangements may be made within the scope of the invention without departing from the spirit thereof, and the invention includes all such substitutions, modifications, additions and/or rearrangements.

### BRIEF DESCRIPTION OF THE DRAWINGS

The following drawing forms part of the present specification and are included to further demonstrate certain aspects of the present invention. The invention may be better understood by reference to one or more of these drawings in combination with the detailed description of specific embodiments presented herein.

FIG. 1 NMO VH1 and VH4 Signature.

### DESCRIPTION OF ILLUSTRATIVE EMBODIMENTS

Since 1998, a number of independent laboratories have documented that VH4-expressing B cells are overrepresented in the CSF (Colombo et al., 2000; Monson et al., 2005; Owens et al., 2003; Qin et al., 1998; Ritchie et al., 2004) and brain lesions (Baranzini et al., 1999; Owens et al., 1998) of MS patients. This finding was initially inconspicuous since clonally expanding and autoreactive B cells from the CSF of MS patients could be found utilizing variable genes from any of the heavy (and light) chain families (Buluwela and Rabbitts, 1988; Humphries et al., 1988; Kodaira et al. 1986; Lee et al., 1987; Shen et al., 1987). However, emerging evidence that the VH4-expressing B cell population harbors autoreactive B cells (Koelsch et al., 2007), combined with the established observation that VH4-expressing B cells are overrepresented in CNS-derived B cell populations from MS patients (Colombo et al., 2000; Owens et al., 2003; Qin et al., 1998; Ritchie et al., 2004; Baranzini et al., 1999; Owens et al., 1998; Harp et al., 2007; Owens et al., 2007), prompted us to question the role of VH4-expressing B cells in the CSF of MS patients.

To address this issue, the inventor previously compared repertoire characteristics from their database of 405 CSF-derived B cells from 13 MS patients to that of healthy controls as well as to several other B cell mediated autoimmune diseases or other CNS-related disorders. The inventor predicted that VH4-expressing B cells from the CSF of MS patients could be enriched for features associated with autoreactivity since (i) VH4 expressing B cells from patients with autoimmune diseases (including SLE and RA) are enriched for autoreactivity (Pugh-Bernard et al., 2001; Zheng et al., 2004; Mockridge et al., 2004; Voswinkel et al., 1997; Hayashi et al., 2007; Huang et al., 1998), and (ii) some autoreactive, clonally-expanded CSF-derived B cells from MS patients use VH4 in their antibody rearrangements (Lambracht-Washington et al., 2007). Features that the inventors was particularly interested in were those known to be associated with autoreactivity including bias towards VH4-34 usage (Zheng et al., 2004) and features associated with receptor editing including bias in JH6 usage and long CDR3 lengths (Zheng et al., 2004; Meffre et al., 2000). Diminished mutational frequency has been associated with receptor editing (Meffre et al., 2000), and diminished mutation targeting has been associated with clonally expanded CSF derived B cell populations in MS patients (Monson et al., 2005) and thus were also included in the analyses.

In order to perform this VH4-specific analysis, the inventor constructed an extensive CSF B cell database containing 405 CSF-derived B cells from 13 MS patients. Overrepresentation of VH4-expressing CD19+ B cells in the CSF of MS patients was unique since VH4 overrepresentation was not observed in B cell repertoires from the peripheral blood of (i) healthy control donors, (ii) patients with other autoimmune diseases with B cell involvement, including systemic lupus erythematosis (SLE) or Sjögren's syndrome, or (iii) MS patients from the same cohort. In fact, in depth analysis of those VH4-expressing B cells from the peripheral blood of MS patients within this cohort indicated that this group of B cells was likely recognized for their autoreactive potential (as evidenced by high JH6 usage and long CDR3 length), and were denied further selection (as evidenced by low mutational frequencies). The inventor also did not observe overrepresentation of VH4-expressing B cells in the CSF of patients with other neurological diseases, indicating that over-representation of VH4-expressing B cells in the CSF of MS patients is not due to bias in the ability of VH4-expressing B cells to enter the CNS. Taken together, these data suggest that VH4-expressing B cells are selected into the CSF B cell repertoire of MS patients in particular, and is further validated by the high mutational frequencies and punctuated mutational targeting observed in this population.

Of the three CIS patients included in this comparison study, even those that convert to CDMS within the next year (CIS429 and CIS03-01) did not have the overrepresentation of VH4 family usage in their CSF-derived CD19+ B cell population. In contrast, evidence of VH4 overrepresentation is observed in the CD138+ plasma cells from CIS03-01. Since the plasma cells and plasma blasts are most likely arising from the CD19+ B cell population (matching clones can be found in both compartments) (Martin Mdel and Monson, 2007), it is reasonable to hypothesize that VH4 expressing B cells which recognize their antigen in the CNS do not linger in the memory pool long, but are signaled to differentiate rapidly into plasma blasts and plasma cells. This hypothesis is also further substantiated by the lack of receptor editing in the VH4 expressing CSF-derived B cells from these patients (as assessed by normal JH6 usage and CDR3 length), as well as documentation that plasmablasts and plasma cells are highly enriched in the CSF of these patients (Cepok et al., 2005; Winges et al., 2007). Dysregulation of these VH4 cells at the initiation of disease processes may be a central component of ongoing pathogenesis.

The inventor expected the increase in VH4 family usage would correspond to an increase in particular VH4 genes used most frequently in MS lesions and in the clones found in MSCSF such as 4-34, 4-39 and 4-59 (Monson et al., 2005; Owens et al., 1998). However, usage frequency of individual VH4 genes within the VH4-expressing CSF B cell subdatabase was no different than in PB of any cohort the inventor analyzed with the exception of VH4-34, which was utilized more frequently in SLE and Sjögren's than in MSCSF. It is possible that B cells from the MS patients examined were responding to a variety of VH4-binding antigens, so that the combination of these made an increase in a single gene indeterminable. Another possibility is an antigen may bind to the VH4 genes and cause a superantigen response in only the B cells expressing VH4, similarly to what is seen with staphylococcal enterotoxin A with VH3-expressing B cells (Domiati-Saad and Lipsky, 1998). However, superantigen binding capacity is diminished with high mutation accumulation (Oppezzo et al., 2004), and so a classical superantigen response is unlikely. In contrast, EBV infected memory B cells tend to have high mutational frequencies and prevalent mutational targeting (Souza et al., 2007) similar to what the inventor described in the MSCSF database presented here, but no mechanism of EBV infection susceptibility or immune response to the virus has been reported that favors VH4-expressing B cells over other heavy chain family expression. Nevertheless, the elevated mutational frequency observed in VH4-expressing B cells from the CSF of MS patients extends the inventor's previous hypothesis that CSF-derived B cells responding to antigen in the CNS are heavily driven within the CNS itself to suggest that much of this heightened activity is occurring within the VH4-expressing CSF-derived B cell populations. Whether these B cells are responding to self-antigens or valid foreign targets remains controversial. However, mutational analysis indicates that the VH4-expressing CSF-derived B cells from MS patients had gone through a typical germinal center, since mutational targeting to CDR and to DGYWWRCH motifs is intact, unlike what has been observed in the individual clonal populations from MS patients in the cohort (Monson et al., 2005). In addition, targeting was actually increased in the MSCSFVH4 subdatabase, most likely because the number of rounds of somatic hypermutation the B cells had undergone in response to antigen was extensive (evidenced by the high mutation frequency). Defining the antigen specificity of highly mutated, VH4-expressing CSF-derived B cells from MS patients will be paramount to resolving the mechanism of this unique selection of VH4 expressing B cells in the CSF of MS patients.

Hyperintense mutation accumulation in the MSCSF database enabled the inventor to identify a unique 5 codon signature of VH4 replacement mutations—codons 31B, 40, 57, 60 and 69—that was not observed in the control databases. Of these 5 codons, 31B was particularly interesting because it accumulated replacement mutations at a rate 7-fold higher than expected, suggesting that this codon plays a pivotal role in antigen-antibody interactions. It is possible that myelin basic protein (MBP) may be excluded from this list of possible antigens interacting with this unique antibody signature since one of the clonally expanded CSF-derived B cells strongly reactive to MBP (Lambracht-Washington et al., 2007) utilized a VH4-59 gene, which does not contain codon 31B. Also, since other databases in this analyses rarely (if ever) accumulated mutations in this position (0.17% in HCPB), it is likely that the antigen targets of VH4 expressing CSF-derived B cells from MS patients are not seen to a great extent in peripheral blood from healthy donors.

Codon composition can also influence the protein structure of antibody variable regions (Chothia et al., 1992). VH4-34 and 4-59 have a similar structure, as they have neither codons 31A or 31B; VH4-04, 4-B, and 4-28 have only codon 31A; and the 4-30 sub-genes, 4-39, 4-61, and 4-31 have both codons 31A and 31B. In addition, several crucial codons are needed to maintain structure; none of the VH4 signature codons are key residues that would change the structure of the antibody (Chothia et al., 1992; Chothia and Lesk, 1987). This infers that genes of similar structure have similar antigen-binding sites, though the exact placement may differ due to the size, hydrophilicity, and polarity of surrounding residues. By the method designated by Clothia et al. (1992), CDR1 is comprised of residues 26 through 32 because these are outside the framework β-sheets and form a loop involved in the antigen binding pocket, and CDR2 is only residues 50 through 58; this translates into codon 30, 31B, 52, 56, and 57 are all in direct contact with the antigen, while 60 is between the antigen binding pocket and another surface loop not directly involved with antigen binding (Chothia et al., 1992). Therefore, codons 30 and 52 are likely “cold,” to maintain efficient antibody interaction with the antigen, while variation in codons 31B (in the few genes it is in), 56, and 57 provide more effective binding to their antigen with different size, hydrophilicity, or polarity properties. It is less clear why residues 40, 69, 81, and 89 are “hot” or residue 68 is “cold,” and how replacement mutations at these positions affect VH4 antigen binding (FIG. 5). Investigating the impact of replacement mutations at these positions will provide important clues regarding the interaction of these VH4 utilizing antibodies with self-antigens in the CNS.

It is also likely that different combinations of residue replacements affect binding to discreet antigens. For example, perhaps the combination of replacements at codons A, B and C mediate high affinity binding to antigen X, while replacements at codons BDE mediate high affinity binding to antigen Y. This would explain the differences in replacement mutation positions in different VH4 genes; codon positions ABC are needed for 4-31 to bind antigen X, while codon positions BDE are needed for 4-39 to bind antigen Y. In support of this, the inventor found that different VH4 genes do selectively use the MS signature mutations at varying levels; for example, VH4-30 has more mutations in codons 56 and 81, while VH4-39 tends to accumulate mutations more rapidly in codons 31B, 50, 56, and 81.

Thus, VH4 family usage is substantially increased in both CD19+ B cells and CD138+ plasma cells isolated from the central nervous system of MS patients (Owens et al., 2007), but as shown here, not in healthy controls, patients with other CNS-related diseases, or patients with other B cell related autoimmune diseases. The VH4 overexpression seen in the MS patients is due to changes in use of many of the genes in the VH4 family (rather than VH4-34 alone), and mutational analysis suggests that antigen-driven selection in the context of classical germinal centers is preserved. Thus, the VH4 expressing B cells from the CSF of MS patients are not dysregulated at this level of selection. More importantly, a unique 11 codon footprint of mutational characteristics can be found in the MSCSF VH4 subdatabase that is not observed in healthy control peripheral blood or CSF-derived B cells from patients with other neurological diseases. This signature, which accumulates replacement mutations up to 7-fold more frequently than in healthy control PB-derived B cells, is most likely a combination of sub-signatures that mediate effective binding to antigens present in the CNS.

The inventor continues to develop the use of this signature to predict or diagnose MS in subjects. As part of this process, the inventor sought to determine whether patients with NMO are distinguishable from patients with MS using this same testing strategy. The inventor considered two possible scenarios. First, that the antibody gene signature associated with MS may also be present in patients with NMO since several clinical features are similar in these two patient types. Second, that the antibody gene signature associated with MS may not be present in patients with NMO, even though several clinical features are similar in these two patient groups. By the same token, it was also possible that NMO patients may exhibit a pattern of somatic hypermutations or “signature” that is unique to NMO and is not expressed by B cells from MS patients.

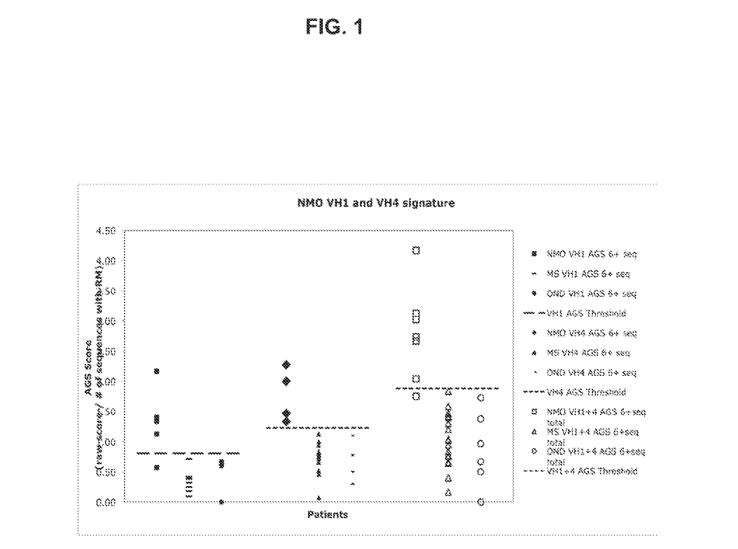

To test this hypothesis, the inventor analyzed NMO antibody gene databases to evaluate whether NMO patients also carried the signature associated with MS. The inventor then compared the somatic hypermuation patterns in the MS antibody database to the somatic hypermuation patterns in the NMO antibody database to see if codons could be identifed that accumulated somatic hypermutations in NMO antibody genes that had not accumulated somatic hypermutations in MS antibody genes. These analyses resulted in two conclusions: (1) the signature associated with MS is expressed by NMO patients; and (2) NMO patients carry a signature that is distinguishable from MS (FIG. 1). The following paragraph details the NMO signature discovery.

The inventor generated a subdatabase from the MS antibody database and from the NMO antibody database that would only include those mutations that had resulted in a codon amino acid replacement. This ensured the analysis would focus on mutations that resulted in a change to the antibody protein itself. Next, the inventor calculated mutation frequencies at each codon position and used chi-square testing to identify codon positions that had mutation frequencies that were statistically different in the MS antibody subdatabase compared to the NMO subdatabase. This analysis led to the identification of 18 codons that had accumulated mutations more frequently in the NMO antibody database in comparison to the MS database. Six of these codons were in genes of the VH1 family, and 12 of these codons were in genes of the VH4 family. An expansion of the antibody gene family designations is provided in the following section.

1. VH1 and VH4

The normal immune system has the ability to generate millions of antibodies with different antigen binding abilities. The diversity is brought about by the complexities of constructing immunoglobulin molecules. These molecules consist of paired polypeptide chains (heavy and light) each containing a constant and a variable region. The structures of the variable regions of the heavy and light chains are specified by immunoglobulin V genes. The heavy chain variable region is derived from three gene segments known as VH, D and JH. In humans there are about 100 different VH segments, over 20 D segments and six JH segments. The light chain genes have only two segments, the VL and JL segments. Antibody diversity is the result of random combinations of VH/D/JH segments with VUJL components superimposed on which are several mechanisms including junctional diversity and somatic mutation.

The germline VH genes can be separated into at least six families (VH1 through VH6) based on DNA nucleotide sequence identity of the first 95 to 101 amino acids. Members of the same family typically have ≧80% sequence identity, whereas members of different families have less than 70% identity. These families range in size from one VH6 gene to an estimated greater than 45 VH3 genes. In addition, many pseudogenes exist. Recent studies have nearly completed a physical map of the VH locus on chromosome 14₈32.l3.l5. It has now been estimated that the human VH repertoire is represented by approximately 50 functional VH segments with about an equal number of pseudogenes. These studies estimate the size of the VH locus to be approximately 1100 kb, which is less than half the previous estimate of 2.5 to 3 megabases as determined by pulse field gel electrophoreis. The VH1 family of genes contains 11 different members: 1-02, 1-03, 1-08, 1-18, 1-24, 1-45, 1-46, 1-58, 1-69, 1-e, 1-f. The VH4 family of genes contains 9 different members: 4-04, 4-28, 4-30, 4-31, 4-34, 4-39, 4-59, 4-61, 4-B4.

A. VH1

The present invention relates to identifcation of a “signature” in the VH1 sequences of certain B cells. The sequence signature initially comprises residues 47, 54, 70, 79, 84, and 91. By examining the sequence at these positions, and identifying mutations at one or more of the positions, it can be determined that a subject is at risk of developing NMO (and not MS) and, in the presence of additional factors, has NMO.

B. VH4 The present invention relates to identifcation of a “signature” in the VH4 sequences of certain B cells. The sequence signature initially comprises residues 36, 39, 45, 46, 50, 59, 61, 65, 67, 70, 86, and 90. By examining the sequence at these positions, and identifying mutations at one or more of the positions, it can be determined that a subject is at risk of developing NMO and, in the presence of additional factors, has NMO.

### II. NUCLEIC ACIDS AND DETECTION METHODS THEREFOR

Another aspect of the present invention concerns isolated DNA segments and their use in detecting the presence of mutations in certain codons of the VH1 and VH4 segments from a subject. Many methods described herein will involve the use of amplification primers, oligonucleotide probes, and other nucleic acid elements involved in the analysis of genomic DNA, cDNA or mRNA transcripts, which is the germline or normal sequence of VH4 family genes which the germline or normal sequence of VH1 family genes.

The term “nucleic acid” is well known in the art. A “nucleic acid” as used herein will generally refer to a molecule (i.e., a strand) of DNA or RNA comprising a nucleobase. A nucleobase includes, for example, a naturally-occurring purine or pyrimidine base found in DNA (e.g., an adenine “A,” a guanine “G,” a thymine “T” or a cytosine “C”) or RNA (e.g., an A, a G, an uracil “U” or a C). The term “nucleic acid” encompass the terms “oligonucleotide” and “polynucleotide,” each as a subgenus of the term “nucleic acid.” The term “oligonucleotide” refers to a molecule of between about 3 and about 100 nucleobases in length. The term “polynucleotide” refers to at least one molecule of greater than about 100 nucleobases in length. A “gene” refers to coding sequence of a gene product, as well as introns and the promoter of the gene product.

These definitions generally refer to a single-stranded molecule, but in specific embodiments will also encompass an additional strand that is partially, substantially or fully complementary to the single-stranded molecule. Thus, a nucleic acid may encompass a double-stranded molecule that comprises complementary strands or “complements” of a particular sequence comprising a molecule. In particular aspects, a nucleic acid encodes a protein or polypeptide, or a portion thereof

A. Preparation of Nucleic Acids

A nucleic acid may be made by any technique known to one of ordinary skill in the art, such as for example, chemical synthesis, enzymatic production or biological production. Non-limiting examples of a synthetic nucleic acid (e.g., a synthetic oligonucleotide), include a nucleic acid made by in vitro chemical synthesis using phosphotriester, phosphite or phosphoramidite chemistry and solid phase techniques such as described in EP 266,032, incorporated herein by reference, or via deoxynucleoside H-phosphonate intermediates as described by Froehler et al., 1986 and U.S. Pat. No. 5,705,629, each incorporated herein by reference. In the methods of the present invention, one or more oligonucleotide may be used. Various different mechanisms of oligonucleotide synthesis have been disclosed in for example, U.S. Pat. Nos. 4,659,774, 4,816,571, 5,141,813, 5,264,566, 4,959,463, 5,428,148, 5,554,744, 5,574,146, 5,602,244, each of which is incorporated herein by reference.

A non-limiting example of an enzymatically produced nucleic acid include one produced by enzymes in amplification reactions such as PCR™ (see for example, U.S. Pat. No. 4,683,202 and U.S. Pat. No. 4,682,195, each incorporated herein by reference), or the synthesis of an oligonucleotide described in U.S. Pat. No. 5,645,897, incorporated herein by reference. A non-limiting example of a biologically produced nucleic acid includes a recombinant nucleic acid produced (i.e., replicated) in a living cell, such as a recombinant DNA vector replicated in bacteria (see for example, Sambrook et al. 2001, incorporated herein by reference).

B. Purification of Nucleic Acids

A nucleic acid may be purified on polyacrylamide gels, cesium chloride centrifugation gradients, chromatography columns or by any other means known to one of ordinary skill in the art (see for example, Sambrook et al., 2001, incorporated herein by reference). In some aspects, a nucleic acid is a pharmacologically acceptable nucleic acid. Pharmacologically acceptable compositions are known to those of skill in the art, and are described herein.

In certain aspects, the present invention concerns a nucleic acid that is an isolated nucleic acid. As used herein, the term “isolated nucleic acid” refers to a nucleic acid molecule (e.g., an RNA or DNA molecule) that has been isolated free of, or is otherwise free of, the bulk of the total genomic and transcribed nucleic acids of one or more cells. In certain embodiments, “isolated nucleic acid” refers to a nucleic acid that has been isolated free of, or is otherwise free of, bulk of cellular components or in vitro reaction components such as for example, macromolecules such as lipids or proteins, small biological molecules, and the like.

C. Nucleic Acid Complements

As discussed above, the present invention encompasses a nucleic acid that is complementary to a nucleic acid. A nucleic acid is “complements” or is “complementary” to another nucleic acid when it is capable of base-pairing with another nucleic acid according to the standard Watson-Crick, Hoogsteen or reverse Hoogsteen binding complementarity rules. As used herein “another nucleic acid” may refer to a separate molecule or a spatial separated sequence of the same molecule. In preferred embodiments, a complement is a hybridization probe or amplification primer for the detection of a nucleic acid polymorphism.

As used herein, the term “complementary” or “complement” also refers to a nucleic acid comprising a sequence of consecutive nucleobases or semiconsecutive nucleobases (e.g., one or more nucleobase moieties are not present in the molecule) capable of hybridizing to another nucleic acid strand or duplex even if less than all the nucleobases do not base pair with a counterpart nucleobase. However, in some diagnostic or detection embodiments, completely complementary nucleic acids are preferred.

D. Nucleic Acid Detection and Evaluation

Those in the art will readily recognize that nucleic acid molecules may be double-stranded molecules and that reference to a particular site on one strand refers, as well, to the corresponding site on a complementary strand. Thus, in defining a polymorphic site, reference to an adenine, a thymine (uridine), a cytosine, or a guanine at a particular site on the plus (sense or coding) strand of a nucleic acid molecule is also intended to include the thymine (uridine), adenine, guanine, or cytosine (respectively) at the corresponding site on a minus (antisense or noncoding) strand of a complementary strand of a nucleic acid molecule. Thus, reference may be made to either strand and still comprise the same polymorphic site and an oligonucleotide may be designed to hybridize to either strand. Throughout the text, in identifying a polymorphic site, reference is made to the sense strand, only for the purpose of convenience.

Typically, the nucleic acid mixture is isolated from a biological sample taken from the individual, such as a blood, fecal or tissue (e.g., intestinal mucosal) sample using standard techniques such as disclosed in Jones (1963) which is hereby incorporated by reference. Other suitable tissue samples include whole blood, saliva, tears, urine, sweat, buccal, skin and hair. The nucleic acid mixture may be comprised of genomic DNA, mRNA, or cDNA. Furthermore it will be understood by the skilled artisan that mRNA or cDNA preparations would not be used to detect polymorphisms located in introns or in 5′ and 3′ non-transcribed regions.

The identity of a nucleotide (or nucleotide pair) at a polymorphic site may be determined by amplifying a target region(s) containing the polymorphic site(s) directly from one or both copies of the gene present in the individual and the sequence of the amplified region(s) determined by conventional methods. It will be readily appreciated by the skilled artisan that only one nucleotide will be detected at a polymorphic site in individuals who are homozygous at that site, while two different nucleotides will be detected if the individual is heterozygous for that site. The polymorphism may be identified directly, known as positive-type identification, or by inference, referred to as negative-type identification. For example, where a SNP is known to be guanine and cytosine in a reference population, a site may be positively determined to be either guanine or cytosine for an individual homozygous at that site, or both guanine and cytosine, if the individual is heterozygous at that site. Alternatively, the site may be negatively determined to be not guanine (and thus cytosinecytosine) or not cytosine (and thus guanineguanine).

The target region(s) may be amplified using any oligonucleotide-directed amplification method, including but not limited to polymerase chain reaction (PCR) (U.S. Pat. No. 4,965,188), ligase chain reaction (LCR) (Barany et al., 1991; WO9001069), and oligonucleotide ligation assay (OLA) (Landegren et al., 1988). Oligonucleotides useful as primers or probes in such methods should specifically hybridize to a region of the nucleic acid that contains or is adjacent to the polymorphic site. Typically, the oligonucleotides are between 10 and 35 nucleotides in length and preferably, between 15 and 30 nucleotides in length. Most preferably, the oligonucleotides are 20 to 25 nucleotides long. The exact length of the oligonucleotide will depend on many factors that are routinely considered and practiced by the skilled artisan.

Other known nucleic acid amplification procedures may be used to amplify the target region including transcription-based amplification systems (U.S. Pat. No. 5,130,238; EP 329,822; U.S. Pat. No. 5,169,766, WO89/06700) and isothermal methods (Walker et al., 1992).

A polymorphism in the target region may also be assayed before or after amplification using one of several hybridization-based methods known in the art. Typically, allele-specific oligonucleotides are utilized in performing such methods. The allele-specific oligonucleotides may be used as differently labeled probe pairs, with one member of the pair showing a perfect match to one variant of a target sequence and the other member showing a perfect match to a different variant. In some embodiments, more than one polymorphic site may be detected at once using a set of allele-specific oligonucleotides or oligonucleotide pairs.

Hybridization of an allele-specific oligonucleotide to a target polynucleotide may be performed with both entities in solution, or such hybridization may be performed when either the oligonucleotide or the target polynucleotide is covalently or noncovalently affixed to a solid support. Attachment may be mediated, for example, by antibody-antigen interactions, poly-L-Lys, streptavidin or avidin-biotin, salt bridges, hydrophobic interactions, chemical linkages, UV cross-linking baking, etc. Allele-specific oligonucleotides may be synthesized directly on the solid support or attached to the solid support subsequent to synthesis. Solid-supports suitable for use in detection methods of the invention include substrates made of silicon, glass, plastic, paper and the like, which may be formed, for example, into wells (as in 96-well plates), slides, sheets, membranes, fibers, chips, dishes, and beads. The solid support may be treated, coated or derivatized to facilitate the immobilization of the allele-specific oligonucleotide or target nucleic acid.

The genotype for one or more polymorphic sites in the gene of an individual may also be determined by hybridization of one or both copies of the gene, or a fragment thereof, to nucleic acid arrays and subarrays such as described in WO 95/1995. The arrays would contain a battery of allele-specific oligonucleotides representing each of the polymorphic sites to be included in the genotype or haplotype.

The identity of polymorphisms may also be determined using a mismatch detection technique, including but not limited to the RNase protection method using riboprobes (Winter et al., 1985; Meyers et al., 1985) and proteins which recognize nucleotide mismatches, such as the E. coli mutS protein (Modrich, 1991). Alternatively, variant alleles can be identified by single strand conformation polymorphism (SSCP) analysis (Orita et al., 1989; Humphries, et al., 1996) or denaturing gradient gel electrophoresis (DGGE) (Wartell et al., 1990; Sheffield et al., 1989).

A polymerase-mediated primer extension method may also be used to identify the polymorphism(s). Several such methods have been described in the patent and scientific literature. Extended primers containing a polymorphism may be detected by mass spectrometry as described in U.S. Pat. No. 5,605,798. Another primer extension method is allele-specific PCR (Ruano et al., 1989; Ruano et al., 1991; WO 9322456; Turki et al., 1995).

1. Hybridization

The use of a probe or primer of between 7, 8, 9, 10, 11, 12, 13, 14, 15, 16, 17, 18, 19, 20, 21, 22, 23, 24, 25, 50, 60, 70, 80, 90, or 100 nucleotides, preferably between 17 and 100 nucleotides in length, or in some aspects of the invention up to 1-2 kilobases or more in length, allows the formation of a duplex molecule that is both stable and selective. Molecules having complementary sequences over contiguous stretches greater than 20 bases in length are generally preferred, to increase stability and/or selectivity of the hybrid molecules obtained. One will generally prefer to design nucleic acid molecules for hybridization having one or more complementary sequences of 20 to 30 nucleotides, or even longer where desired. Such fragments may be readily prepared, for example, by directly synthesizing the fragment by chemical means or by introducing selected sequences into recombinant vectors for recombinant production.

Accordingly, the nucleotide sequences of the invention may be used for their ability to selectively form duplex molecules with complementary stretches of DNAs and/or RNAs or to provide primers for amplification of DNA or RNA from samples. Depending on the application envisioned, one would desire to employ varying conditions of hybridization to achieve varying degrees of selectivity of the probe or primers for the target sequence.

For applications requiring high selectivity, one will typically desire to employ relatively high stringency conditions to form the hybrids. For example, relatively low salt and/or high temperature conditions, such as provided by about 0.02 M to about 0.10 M NaCl at temperatures of about 50° C. to about 70° C. Such high stringency conditions tolerate little, if any, mismatch between the probe or primers and the template or target strand and would be particularly suitable for isolating specific genes or for detecting a specific polymorphism. It is generally appreciated that conditions can be rendered more stringent by the addition of increasing amounts of formamide. For example, under highly stringent conditions, hybridization to filter-bound DNA may be carried out in 0.5 M NaHPO₄, 7% sodium dodecyl sulfate (SDS), 1 mM EDTA at 65° C., and washing in 0.1×SSC0.1% SDS at 68° C. (Ausubel et al., 1989).

Conditions may be rendered less stringent by increasing salt concentration and/or decreasing temperature. For example, a medium stringency condition could be provided by about 0.1 to 0.25 M NaCl at temperatures of about 37° C. to about 55° C., while a low stringency condition could be provided by about 0.15 M to about 0.9 M salt, at temperatures ranging from about 20° C. to about 55° C. Under low stringent conditions, such as moderately stringent conditions the washing may be carried out for example in 0.2×SSC0.1% SDS at 42° C. (Ausubel et al., 1989). Hybridization conditions can be readily manipulated depending on the desired results.

In other embodiments, hybridization may be achieved under conditions of, for example, 50 mM Tris-HCl (pH 8.3), 75 mM KCl, 3 mM MgCl₂, 1.0 mM dithiothreitol, at temperatures between approximately 20° C. to about 37° C. Other hybridization conditions utilized could include approximately 10 mM Tris-HCl (pH 8.3), 50 mM KCl, 1.5 mM MgCl₂, at temperatures ranging from approximately 40° C. to about 72° C.

In certain embodiments, it will be advantageous to employ nucleic acids of defined sequences of the present invention in combination with an appropriate means, such as a label, for determining hybridization. A wide variety of appropriate indicator means are known in the art, including fluorescent, radioactive, enzymatic or other ligands, such as avidin/biotin, which are capable of being detected. In preferred embodiments, one may desire to employ a fluorescent label or an enzyme tag such as urease, alkaline phosphatase or peroxidase, instead of radioactive or other environmentally undesirable reagents. In the case of enzyme tags, colorimetric indicator substrates are known that can be employed to provide a detection means that is visibly or spectrophotometrically detectable, to identify specific hybridization with complementary nucleic acid containing samples. In other aspects, a particular nuclease cleavage site may be present and detection of a particular nucleotide sequence can be determined by the presence or absence of nucleic acid cleavage.

In general, it is envisioned that the probes or primers described herein will be useful as reagents in solution hybridization, as in PCR, for detection of expression or genotype of corresponding genes, as well as in embodiments employing a solid phase. In embodiments involving a solid phase, the test DNA (or RNA) is adsorbed or otherwise affixed to a selected matrix or surface. This fixed, single-stranded nucleic acid is then subjected to hybridization with selected probes under desired conditions. The conditions selected will depend on the particular circumstances (depending, for example, on the G+C content, type of target nucleic acid, source of nucleic acid, size of hybridization probe, etc.). Optimization of hybridization conditions for the particular application of interest is well known to those of skill in the art. After washing of the hybridized molecules to remove non-specifically bound probe molecules, hybridization is detected, and/or quantified, by determining the amount of bound label. Representative solid phase hybridization methods are disclosed in U.S. Pat. Nos. 5,843,663, 5,900,481 and 5,919,626. Other methods of hybridization that may be used in the practice of the present invention are disclosed in U.S. Pat. Nos. 5,849,481, 5,849,486 and 5,851,772. The relevant portions of these and other references identified in this section of the Specification are incorporated herein by reference.

2. Amplification of Nucleic Acids

Nucleic acids used as a template for amplification may be isolated from cells, tissues or other samples according to standard methodologies (Sambrook et al., 2001). In certain embodiments, analysis is performed on whole cell or tissue homogenates or biological fluid samples with or without substantial purification of the template nucleic acid. The nucleic acid may be genomic DNA or fractionated or whole cell RNA. Where RNA is used, it may be desired to first convert the RNA to a complementary DNA.

The term “primer,” as used herein, is meant to encompass any nucleic acid that is capable of priming the synthesis of a nascent nucleic acid in a template-dependent process. Typically, primers are oligonucleotides from ten to twenty and/or thirty base pairs in length, but longer sequences can be employed. Primers may be provided in double-stranded and/or single-stranded form, although the single-stranded form is preferred.

Pairs of primers designed to selectively hybridize to nucleic acids corresponding to the variable heavy chain gene locus, variants and fragments thereof are contacted with the template nucleic acid under conditions that permit selective hybridization. Depending upon the desired application, high stringency hybridization conditions may be selected that will only allow hybridization to sequences that are completely complementary to the primers. In other embodiments, hybridization may occur under reduced stringency to allow for amplification of nucleic acids that contain one or more mismatches with the primer sequences. Once hybridized, the template-primer complex is contacted with one or more enzymes that facilitate template-dependent nucleic acid synthesis. Multiple rounds of amplification, also referred to as “cycles,” are conducted until a sufficient amount of amplification product is produced.

The amplification product may be detected, analyzed or quantified. In certain applications, the detection may be performed by visual means. In certain applications, the detection may involve indirect identification of the product via chemiluminescence, radioactive scintigraphy of incorporated radiolabel or fluorescent label or even via a system using electrical and/or thermal impulse signals (Affymax technology; Bellus, 1994).

A number of template dependent processes are available to amplify the oligonucleotide sequences present in a given template sample. One of the best known amplification methods is the polymerase chain reaction (referred to as PCR™) which is described in detail in U.S. Pat. Nos. 4,683,195, 4,683,202 and 4,800,159, and in Innis et al., 1988, each of which is incorporated herein by reference in their entirety.

Primer extension, which may be used as a stand alone technique or in combination with other methods (such as PCR), requires a labeled primer (usually 20-50 nucleotides in length) complementary to a region near the 5′ end of the gene. The primer is allowed to anneal to the RNA and reverse transcriptase is used to synthesize complementary cDNA to the RNA until it reaches the 5′ end of the RNA.

Another method for amplification is ligase chain reaction (“LCR”), disclosed in European Application No. 320 308, incorporated herein by reference in its entirety. U.S. Pat. No. 4,883,750 describes a method similar to LCR for binding probe pairs to a target sequence. A method based on PCR™ and oligonucleotide ligase assay (OLA) (described in further detail below), disclosed in U.S. Pat. No. 5,912,148, may also be used.

Alternative methods for amplification of target nucleic acid sequences that may be used in the practice of the present invention are disclosed in U.S. Pat. Nos. 5,843,650, 5,846,709, 5,846,783, 5,849,546, 5,849,497, 5,849,547, 5,858,652, 5,866,366, 5,916,776, 5,922,574, 5,928,905, 5,928,906, 5,932,451, 5,935,825, 5,939,291 and 5,942,391, Great Britain Application 2 202 328, and in PCT Application PCTUS8901025, each of which is incorporated herein by reference in its entirety. Qbeta Replicase, described in PCT Application PCTUS8700880, may also be used as an amplification method in the present invention.

An isothermal amplification method, in which restriction endonucleases and ligases are used to achieve the amplification of target molecules that contain nucleotide 5′-[alpha-thio]-triphosphates in one strand of a restriction site may also be useful in the amplification of nucleic acids in the present invention (Walker et al., 1992). Strand Displacement Amplification (SDA), disclosed in U.S. Pat. No. 5,916,779, is another method of carrying out isothermal amplification of nucleic acids which involves multiple rounds of strand displacement and synthesis, i.e., nick translation

Other nucleic acid amplification procedures include transcription-based amplification systems (TAS), including nucleic acid sequence based amplification (NASBA) and 3SR (Kwoh et al., 1989; PCT Application WO 88/0315, incorporated herein by reference in their entirety). European Application 329 822 disclose a nucleic acid amplification process involving cyclically synthesizing single-stranded RNA (“ssRNA”), ssDNA, and double-stranded DNA (dsDNA), which may be used in accordance with the present invention.

PCT Application WO 89/06700 (incorporated herein by reference in its entirety) disclose a nucleic acid sequence amplification scheme based on the hybridization of a promoter regionprimer sequence to a target single-stranded DNA (“ssDNA”) followed by transcription of many RNA copies of the sequence. This scheme is not cyclic, i.e., new templates are not produced from the resultant RNA transcripts. Other amplification methods include “RACE” and “one-sided PCR” (Frohman, 1990; Ohara et al., 1989).

Real-time polymerase chain reaction, also called quantitative real time polymerase chain reaction (qPCR) or kinetic polymerase chain reaction, is a laboratory technique based on the polymerase chain reaction, which is used to amplify and simultaneously quantify a targeted DNA molecule. It enables both detection and quantification (as absolute number of copies or relative amount when normalized to DNA input or additional normalizing genes) of a specific sequence in a DNA sample.

The procedure follows the general principle of polymerase chain reaction; its key feature is that the amplified DNA is quantified as it accumulates in the reaction in real time after each amplification cycle. Two common methods of quantification are the use of fluorescent dyes that intercalate with double-stranded DNA, and modified DNA oligonucleotide probes that fluoresce when hybridized with a complementary DNA.

Frequently, real-time polymerase chain reaction is combined with reverse transcription polymerase chain reaction to quantify low abundance messenger RNA (mRNA), enabling a researcher to quantify relative gene expression at a particular time, or in a particular cell or tissue type. Although real-time quantitative polymerase chain reaction is often marketed as RT-PCR, it should not be confused with reverse transcription polymerase chain reaction, also known as RT-PCR.

A DNA-binding dye binds to all double-stranded (ds)DNA in a PCR reaction, causing fluorescence of the dye. An increase in DNA product during PCR therefore leads to an increase in fluorescence intensity and is measured at each cycle, thus allowing DNA concentrations to be quantified. However, dsDNA dyes such as SYBR Green will bind to all dsDNA PCR products, including non-specific PCR products (such as “primer dimers”). This can potentially interfere with or prevent accurate quantification of the intended target sequence. The reaction is prepared as usual, with the addition of fluorescent dsDNA dye.

The reaction is run in a thermocycler, and after each cycle, the levels of fluorescence are measured with a detector; the dye only fluoresces when bound to the dsDNA (i.e., the PCR product). With reference to a standard dilution, the dsDNA concentration in the PCR can be determined.

Like other real-time PCR methods, the values obtained do not have absolute units associated with it (i.e. mRNA copiescell). As described above, a comparison of a measured DNARNA sample to a standard dilution will only give a fraction or ratio of the sample relative to the standard, allowing only relative comparisons between different tissues or experimental conditions. To ensure accuracy in the quantification, it is usually necessary to normalize expression of a target gene to a stably expressed gene. This can correct possible differences in RNA quantity or quality across experimental samples.

Using fluorescent reporter probes is the most accurate and most reliable of the methods, but also the most expensive. It uses a sequence-specific RNA or DNA-based probe to quantify only the DNA containing the probe sequence; therefore, use of the reporter probe significantly increases specificity, and allows quantification even in the presence of some non-specific DNA amplification. This potentially allows for multiplexing—assaying for several genes in the same reaction by using specific probes with different-coloured labels, provided that all genes are amplified with similar efficiency.

It is commonly carried out with an RNA-based probe with a fluorescent reporter at one end and a quencher of fluorescence at the opposite end of the probe. The close proximity of the reporter to the quencher prevents detection of its fluorescence; breakdown of the probe by the 5′ to 3′ exonuclease activity of the taq polymerase breaks the reporter-quencher proximity and thus allows unquenched emission of fluorescence, which can be detected. An increase in the product targeted by the reporter probe at each PCR cycle therefore causes a proportional increase in fluorescence due to the breakdown of the probe and release of the reporter.

The PCR reaction is prepared as usual (see PCR), and the reporter probe is added. As the reaction commences, during the annealing stage of the PCR both probe and primers anneal to the DNA target. Polymerisation of a new DNA strand is initiated from the primers, and once the polymerase reaches the probe, its 5′-3-exonuclease degrades the probe, physically separating the fluorescent reporter from the quencher, resulting in an increase in fluorescence.

Fluorescence is detected and measured in the real-time PCR thermocycler, and its geometric increase corresponding to exponential increase of the product is used to determine the threshold cycle (C_{T}) in each reaction.

Quantitating gene expression by traditional methods presents several problems. Firstly, detection of mRNA on a Northern blot or PCR products on a gel or Southern blot is time-consuming and does not allow precise quantitation. Also, over the 20-40 cycles of a typical PCR reaction, the amount of product reaches a plateau determined more by the amount of primers in the reaction mix than by the input templatesample.

Relative concentrations of DNA present during the exponential phase of the reaction are determined by plotting fluorescence against cycle number on a logarithmic scale (so an exponentially increasing quantity will give a straight line). A threshold for detection of fluorescence above background is determined. The cycle at which the fluorescence from a sample crosses the threshold is called the cycle threshold, Cₜ. Since the quantity of DNA doubles every cycle during the exponential phase, relative amounts of DNA can be calculated, e.g., a sample whose Cₜ is 3 cycles earlier than another's has 2³=8 times more template.

Amounts of RNA or DNA are then determined by comparing the results to a standard curve produced by RT-PCR of serial dilutions (e.g., undiluted, 1:4, 1:16, 1:64) of a known amount of RNA or DNA. As mentioned above, to accurately quantify gene expression, the measured amount of RNA from the gene of interest is divided by the amount of RNA from a housekeeping gene measured in the same sample to normalize for possible variation in the amount and quality of RNA between different samples. This normalization permits accurate comparison of expression of the gene of interest between different samples, provided that the expression of the reference (housekeeping) gene used in the normalization is very similar across all the samples. Choosing a reference gene fulfilling this criterion is therefore of high importance, and often challenging, because only very few genes show equal levels of expression across a range of different conditions or tissues.

3. Detection of Nucleic Acids

Following any amplification, it may be desirable to separate the amplification product from the template and/or the excess primer. In one embodiment, amplification products are separated by agarose, agarose-acrylamide or polyacrylamide gel electrophoresis using standard methods (Sambrook et al., 2001). Separated amplification products may be cut out and eluted from the gel for further manipulation. Using low melting point agarose gels, the separated band may be removed by heating the gel, followed by extraction of the nucleic acid.

Separation of nucleic acids may also be effected by spin columns and/or chromatographic techniques known in art. There are many kinds of chromatography which may be used in the practice of the present invention, including adsorption, partition, ion-exchange, hydroxylapatite, molecular sieve, reverse-phase, column, paper, thin-layer, and gas chromatography as well as HPLC.

In certain embodiments, the amplification products are visualized, with or without separation. A typical visualization method involves staining of a gel with ethidium bromide and visualization of bands under UV light. Alternatively, if the amplification products are integrally labeled with radio- or fluorometrically-labeled nucleotides, the separated amplification products can be exposed to x-ray film or visualized under the appropriate excitatory spectra.

In one embodiment, following separation of amplification products, a labeled nucleic acid probe is brought into contact with the amplified marker sequence. The probe preferably is conjugated to a chromophore but may be radiolabeled. In another embodiment, the probe is conjugated to a binding partner, such as an antibody or biotin, or another binding partner carrying a detectable moiety.

In particular embodiments, detection is by Southern blotting and hybridization with a labeled probe. The techniques involved in Southern blotting are well known to those of skill in the art (see Sambrook et al., 2001). One example of the foregoing is described in U.S. Pat. No. 5,279,721, incorporated by reference herein, which discloses an apparatus and method for the automated electrophoresis and transfer of nucleic acids. The apparatus permits electrophoresis and blotting without external manipulation of the gel and is ideally suited to carrying out methods according to the present invention.

Other methods of nucleic acid detection that may be used in the practice of the instant invention are disclosed in U.S. Pat. Nos. 5,840,873, 5,843,640, 5,843,651, 5,846,708, 5,846,717, 5,846,726, 5,846,729, 5,849,487, 5,853,990, 5,853,992, 5,853,993, 5,856,092, 5,861,244, 5,863,732, 5,863,753, 5,866,331, 5,905,024, 5,910,407, 5,912,124, 5,912,145, 5,919,630, 5,925,517, 5,928,862, 5,928,869, 5,929,227, 5,932,413 and 5,935,791, each of which is incorporated herein by reference.

4. Other Assays

Other methods for genetic screening may be used within the scope of the present invention, for example, to detect mutations in genomic DNA, cDNA and/or RNA samples. Methods used to detect point mutations include denaturing gradient gel electrophoresis (DGGE), restriction fragment length polymorphism analysis (RFLP), chemical or enzymatic cleavage methods, direct sequencing of target regions amplified by PCR™ (see above), single-strand conformation polymorphism analysis (SSCP) and other methods well known in the art.

One method of screening for point mutations is based on RNase cleavage of base pair mismatches in RNADNA or RNARNA heteroduplexes. As used herein, the term “mismatch” is defined as a region of one or more unpaired or mispaired nucleotides in a double-stranded RNARNA, RNADNA or DNADNA molecule. This definition thus includes mismatches due to insertiondeletion mutations, as well as single or multiple base point mutations.

U.S. Pat. No. 4,946,773 describes an RNase A mismatch cleavage assay that involves annealing single-stranded DNA or RNA test samples to an RNA probe, and subsequent treatment of the nucleic acid duplexes with RNase A. For the detection of mismatches, the single-stranded products of the RNase A treatment, electrophoretically separated according to size, are compared to similarly treated control duplexes. Samples containing smaller fragments (cleavage products) not seen in the control duplex are scored as positive.

Other investigators have described the use of RNase I in mismatch assays. The use of RNase I for mismatch detection is described in literature from Promega Biotech. Promega markets a kit containing RNase I that is reported to cleave three out of four known mismatches. Others have described using the MutS protein or other DNA-repair enzymes for detection of single-base mismatches.

Alternative methods for detection of deletion, insertion or substitution mutations that may be used in the practice of the present invention are disclosed in U.S. Pat. Nos. 5,849,483, 5,851,770, 5,866,337, 5,925,525 and 5,928,870, each of which is incorporated herein by reference in its entirety.

5. Polymorphic Nucleic Acid Screening Methods

Spontaneous mutations that arise during the course of evolution in the genomes of organisms are often not immediately transmitted throughout all of the members of the species, thereby creating polymorphic alleles that co-exist in the species populations. Often polymorphisms are the cause of genetic diseases. Several classes of polymorphisms have been identified. For example, variable nucleotide type polymorphisms (VNTRs), arise from spontaneous tandem duplications of di- or trinucleotide repeated motifs of nucleotides. If such variations alter the lengths of DNA fragments generated by restriction endonuclease cleavage, the variations are referred to as restriction fragment length polymorphisms (RFLPs). RFLPs are been widely used in human and animal genetic analyses.

Another class of polymorphisms is generated by the replacement of a single nucleotide. Such single nucleotide polymorphisms (SNPs) rarely result in changes in a restriction endonuclease site. Thus, SNPs are rarely detectable restriction fragment length analysis. SNPs are the most common genetic variations and occur once every 100 to 300 bases and several SNP mutations have been found that affect a single nucleotide in a protein-encoding gene in a manner sufficient to actually cause a genetic disease. SNP diseases are exemplified by hemophilia, sickle-cell anemia, hereditary hemochromatosis, late-onset Alzheimer's disease, etc.

Several methods have been developed to screen polymorphisms and some examples are listed below. The reference of Kwok and Chen (2003) and Kwok (2001) provide overviews of some of these methods; both of these references are specifically incorporated by reference. SNPs can be characterized by the use of any of these methods or suitable modification thereof. Such methods include the direct or indirect sequencing of the site, the use of restriction enzymes where the respective alleles of the site create or destroy a restriction site, the use of allele-specific hybridization probes, the use of antibodies that are specific for the proteins encoded by the different alleles of the polymorphism, or any other biochemical interpretation.

i. DNA Sequencing

The most commonly used method of characterizing a polymorphism is direct DNA sequencing of the genetic locus that flanks and includes the polymorphism. Such analysis can be accomplished using either the “dideoxy-mediated chain termination method,” also known as the “Sanger Method” (Sanger et al., 1975) or the “chemical degradation method,” also known as the “Maxam-Gilbert method” (Maxam et al., 1977). Sequencing in combination with genomic sequence-specific amplification technologies, such as the polymerase chain reaction may be utilized to facilitate the recovery of the desired genes (Mullis et al., 1986; European Patent Application 50,424; European Patent Application. 84,796, European Patent Application 258,017, European Patent Application. 237,362; European Patent Application. 201,184; U.S. Pat. Nos. 4,683,202; 4,582,788; and 4,683,194), all of the above incorporated herein by reference.

ii. Exonuclease Resistance

Other methods that can be employed to determine the identity of a nucleotide present at a polymorphic site utilize a specialized exonuclease-resistant nucleotide derivative (U.S. Pat. No. 4,656,127). A primer complementary to an allelic sequence immediately 3′- to the polymorphic site is hybridized to the DNA under investigation. If the polymorphic site on the DNA contains a nucleotide that is complementary to the particular exonucleotide-resistant nucleotide derivative present, then that derivative will be incorporated by a polymerase onto the end of the hybridized primer. Such incorporation makes the primer resistant to exonuclease cleavage and thereby permits its detection. As the identity of the exonucleotide-resistant derivative is known one can determine the specific nucleotide present in the polymorphic site of the DNA.

iii. Microsequencing Methods

Several other primer-guided nucleotide incorporation procedures for assaying polymorphic sites in DNA have been described (Komher et al., 1989; Sokolov, 1990; Syvanen 1990; Kuppuswamy et al., 1991; Prezant et al., 1992; Ugozzoll et al., 1992; Nyren et al., 1993). These methods rely on the incorporation of labeled deoxynucleotides to discriminate between bases at a polymorphic site. As the signal is proportional to the number of deoxynucleotides incorporated, polymorphisms that occur in runs of the same nucleotide result in a signal that is proportional to the length of the run (Syvanen et al., 1990).

iv. Extension in Solution

French Patent 2,650,840 and PCT Application WO9102087 discuss a solution-based method for determining the identity of the nucleotide of a polymorphic site. According to these methods, a primer complementary to allelic sequences immediately 3′- to a polymorphic site is used. The identity of the nucleotide of that site is determined using labeled dideoxynucleotide derivatives which are incorporated at the end of the primer if complementary to the nucleotide of the polymorphic site.

v. Genetic Bit Analysis or Solid-Phase Extension

PCT Application WO92/5712 describes a method that uses mixtures of labeled terminators and a primer that is complementary to the sequence 3′ to a polymorphic site. The labeled terminator that is incorporated is complementary to the nucleotide present in the polymorphic site of the target molecule being evaluated and is thus identified. Here the primer or the target molecule is immobilized to a solid phase.

vi. Oligonucleotide Ligation Assay (OLA)

This is another solid phase method that uses different methodology (Landegren et al., 1988). Two oligonucleotides, capable of hybridizing to abutting sequences of a single strand of a target DNA are used. One of these oligonucleotides is biotinylated while the other is detectably labeled. If the precise complementary sequence is found in a target molecule, the oligonucleotides will hybridize such that their termini abut, and create a ligation substrate. Ligation permits the recovery of the labeled oligonucleotide by using avidin. Other nucleic acid detection assays, based on this method, combined with PCR have also been described (Nickerson et al., 1990). Here, PCR is used to achieve the exponential amplification of target DNA, which is then detected using the OLA.

vii. Ligase/Polymerase-Mediated Genetic Bit Analysis

U.S. Pat. No. 5,952,174 describes a method that also involves two primers capable of hybridizing to abutting sequences of a target molecule. The hybridized product is formed on a solid support to which the target is immobilized. Here the hybridization occurs such that the primers are separated from one another by a space of a single nucleotide. Incubating this hybridized product in the presence of a polymerase, a ligase, and a nucleoside triphosphate mixture containing at least one deoxynucleoside triphosphate allows the ligation of any pair of abutting hybridized oligonucleotides. Addition of a ligase results in two events required to generate a signal, extension and ligation. This provides a higher specificity and lower “noise” than methods using either extension or ligation alone and unlike the polymerase-based assays, this method enhances the specificity of the polymerase step by combining it with a second hybridization and a ligation step for a signal to be attached to the solid phase.

viii. Invasive Cleavage Reactions

Invasive cleavage reactions can be used to evaluate cellular DNA for a particular polymorphism. A technology called INVADER® employs such reactions (e.g., de Arruda et al., 2002; Stevens et al., 2003, which are incorporated by reference). Generally, there are three nucleic acid molecules: 1) an oligonucleotide upstream of the target site (“upstream oligo”), 2) a probe oligonucleotide covering the target site (“probe”), and 3) a single-stranded DNA with the target site (“target”). The upstream oligo and probe do not overlap but they contain contiguous sequences. The probe contains a donor fluorophore, such as fluoroscein, and an acceptor dye, such as Dabcyl. The nucleotide at the 3′ terminal end of the upstream oligo overlaps (“invades”) the first base pair of a probe-target duplex. Then the probe is cleaved by a structure-specific 5′ nuclease causing separation of the fluorophorequencher pair, which increases the amount of fluorescence that can be detected. See Lu et al. (2004). In some cases, the assay is conducted on a solid-surface or in an array format.

### III. PREDICTING AND DIAGNOSING NEUROMYELITIS OPTICA

A. Neuromyelitis Optica

The main symptoms of NMO are loss of vision and spinal cord function. As for other etiologies of optic neuritis, the visual impairment usually manifests as decreased visual acuity, although visual field defects, or loss of color vision may occur in isolation or prior to formal loss of acuity. Spinal cord dysfunction can lead to muscle weakness, reduced sensation, or loss of bladder and bowel control. The typical patient has an acute and severe spastic weakness of the legs (paraparesis) or all four limbs (tetraparesis) with sensory signs, often accompanied by loss of bladder control.

As discussed above, NMO is similar to MS in that the body's immune system attacks the myelin surrounding nerve cells. Unlike standard MS, the attacks are not believed to be mediated by the immune system's T cells but rather by antibodies called NMO-IgG, or simply NMO antibodies. These antibodies target a protein called aquaporin 4 in the cell membranes of astrocytes, which acts as a channel for the transport of water across the cell membrane. Aquaporin 4 is found in the processes of the astrocytes that surround the blood-brain barrier, a system responsible for preventing substances in the blood from crossing into the brain. The blood-brain barrier is weakened in NMO, but it is currently unknown how the NMO-IgG immune response leads to demyelination.

Most research into the pathology of NMO has focused on the spinal cord. The damage in the spinal cord can range from inflammatory demyelination to necrotic damage of the white and grey matter. The inflammatory lesions in NMO have been classified as type II lesions (complement mediated demyelinization), but they differ from MS pattern II lesions in their prominent perivascular distribution. Therefore, the pattern of inflammation is often quite distinct from that seen in MS.

Approximately 20% of patients with monophasic NMO have permanent visual loss and 30% have permanent paralysis in one or more legs. Among patients with relapsing NMO, 50% have paralysis or blindness within 5 years. In some patients (33% in one study), transverse myelitis in the cervical spinal cord resulted in respiratory failure and subsequent death. However, the spectrum of NMO has widened due to improved diagnostic criteria, and the options for treatment have improved; as a result, researchers believe that these estimates will be lowered.

The prevalence and incidence of NMO has not been established partly because the disease is underrecognized and often confused with MS. NMO is more common in women than men, with women comprising over 23 of patients and more than 80% of those with the relapsing form of the disease. NMO is more common in Asiatic people than Caucasians. In fact, Asian optic-spinal MS (which constitutes 30% of the cases of MS in Japan) has been suggested to be identical to NMO (differences between optic-spinal and classic MS in Japanese patients). In the indigenous populations of tropical and subtropical regions, MS is rare, but when it appears it often takes the form of optic-spinal MS. The majority of NMO patients have no affected relatives, and it is generally regarded as a non-familial condition.

B. Traditional Diagnosis

The Mayo Clinic proposed a revised set of criteria for diagnosis of NMO in 2006. The new guidelines for diagnosis require two absolute criteria plus at least two of three supportive criteria being:

Absolute criteria:
Optic neuritis
Acute myelitis

Supportive Criteria:
Brain MRI not meeting criteria for MS at disease onset
Spinal cord MRI with contiguous T2-weighted signal abnormality extending over 3 or more vertebral segments, indicating a relatively large lesion in the spinal cord

NMO-IgG Seropositive Status:
The NMO-IgG test checks the existence of antibodies against the aquaporin 4 antigen
   After the development of the NMO-IgG test, the spectrum of disorders that comprise NMO was expanded. The NMO spectrum is now believed to consist of:
Standard NMO, according to the diagnostic criteria described above
Limited forms of NMO, such as single or recurrent events of longitudinally extensive myelitis, and bilateral simultaneous or recurrent optic neuritis
Asian optic-spinal MS. This variant can present CNS involvement like MS
Longitudinally extensive myelitis or optic neuritis associated with systemic auto-immune disease
Optic neuritis or myelitis associated with lesions in specific brain areas such as the hypothalamus, periventricular nucleus, and brainstem
   Whether NMO is a distinct disease or part of the wide spectrum of multiple sclerosis is debated. Recently it has been found that antiviral immune response distinguishes MS and NMO, but being MS an heterogeneous condition, as hepatitis or diabetes are, it is still possible to consider NMO part of the MS spectrum.

NMO has been associated with many systemic diseases, based on anecdoctal evidence of some NMO patients with a comorbid condition. Such conditions include: collagen vascular diseases, autoantibody syndromes, infections with varicella-zoster virus, Epstein-Barr virus, and HIV, and exposure to clioquinol and antituberculosis drugs.

C. Samples and Preparation

The present invention contemplates the identification of VH1 and VH4 sequences from B cells obtained from any sample (fluid or tissue) that would contain such cells. In particular, the present invention will rely on peripheral blood as a source of B cells, given the ease of obtention and the plentiful nature of B cells. In addition, given the CNS implications of NMO, cerebrospinal fluid provides another potential source of B cells for analysis. Methods for separating and analyzing nucleic acids are provided above.

D. Therapy and Prophylaxis

It may be that, on the basis of the diagnosis or prediction provided by the methods described herein, one will wish to begin, end or modify a therapeutic regimen. In particular, subjects diagnosed as having or at risk of developing NMO may be started on a therapeutic regimen. The primary aims of therapy are returning function after an attack, preventing new attacks, and preventing disability. As with any medical treatment, medications used in the management of NMO have several adverse effects, and many possible therapies are still under investigation.

Currently, there is no cure for NMO, but symptoms can be treated. Some patients recover, but many are left with impairment of vision and limbs, which can be severe. Attacks are treated with short courses of high dosage intravenous corticosteroids such as methylprednisolone IV. When attacks progress or do not respond to corticosteroid treatment, plasmapheresis can be an effective treatment. Clinical trials for these treatments contain very small numbers, and most are uncontrolled.

No controlled trials have established the effectiveness of treatments for the prevention of attacks. Many clinicians agree that long-term immunosuppression is required to reduce the frequency and severity of attacks, while others argue the exact opposite. Commonly used immunosuppressant treatments include azathioprine (Imuran) plus prednisone, mycophenolate mofetil plus prednisone, Rituximab, Mitoxantrone, intravenous immunoglobulin (IVIG), and Cyclophosphamide. The monoclonal antibody rituximab is under study. In 2007, NMO was reported to be responsive to glatiramer acetate and to low-dose corticosteroids. Normally, there is some measure of improvement in a few weeks, but residual signs and disability may persist, sometimes severely.

The disease can be monophasic, i.e., a single episode with permanent remission. However, at least 85% of patients have a relapsing form of the disease with repeated attacks of transverse myelitis and/or optic neuritis. In patients with the monophasic form the transverse myelitis and optic neuritis occur simultaneously or within days of each other. On the other hand, patients with the relapsing form are more likely to have weeks or months between the initial attacks and to have better motor recovery after the initial transverse myelitis event. Relapses usually occur early with about 55% of patients having a relapse in the first year and 90% in the first 5 years. Unlike multiple sclerosis, NMO rarely has a secondary progressive phase in which patients have increasing neurologic decline between attacks without remission. Instead, disabilities arise from the acute attacks.

The present invention also contemplates the use of novel therapeutic agents antibodies or peptidespeptoids that bind to the altered VH1/VH4 genes described herein to treat NMO. VH1/VH4-antibody therapeutics can be prepared and screened for reactivity using well-known techniques. Peptides and peptoids that act as “mimotopes,” or epitope-mimicking structures can be administered and used to sequester the VH1/VH4 products away from pathologic interactions. See Reimer & Jensen-Jarolim (2007).

### IV. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials and Methods

NMO antibody databases were obtained from Jeff Bennett, an NIH investigator from University of Colorado-Denver who published an analysis of these repertoires in 2009 (Bennett et al., Ann Neural. 2009 November; 66(5):617-29). The sequence databases were subjected to antibody genetic analysis as the inventor had previously established with the MS-AGS, with the exception that new software (whose design was implemented by Dr. Lindsay Cowell, UTSWMC) was used to assemble the appropriate Excel-based spreadsheets required to perform the final analysis. The final analysis was done by first excluding any mutations in the combined pool of antibody genes from NMO patients that did not result in a replacement of the amino acid at that codon position. Next, mutation frequencies were calculated at each codon position and compared to the mutation frequencies at each codon position in our MS antibody database. Any codon position that had a higher mutation frequency in the NMO antibody database compared to the MS antibody database was called out to a separate list. This separate list was then compared to our healthy control antibody database. Any codon position that maintained a higher mutation frequency in the NMO antibody dabase compared to the healthy control antibody database was called out to the final list of codons that had higher mutation accumulation than either MS antibody genes or healthy control antibody genes. These codons are the framework of the NMO-specific antibody gene signature (AGS-NMO). The inventor then calculated scores for each patient, just as the inventor had done with the AGS-MS, but using this set of codons to generate the values.

### Example 2

### Results

AGS-NMO Using VH4 Genes Only.

This analysis resulted in 12 codons from VH4 antibody genes that had accumulated mutations to a statistically higher magnitude in NMO antibody databases compared to MS or HC antibody databases. The VH4 codons are: 36; 39; 45; 46; 50; 59; 61; 65; 67; 70; 86; 90. The average AGS-NMO score for the NMO cohort is 1.681 (range 1.33 to 2.27). The average AGS-NMO score for the MS cohort is 0.731 (range 0.07 to 1.13). The average AGS-NMO score for the HC cohort is 0.669 (range 0.30 to 1.10). As expected, AGS-NMO scores were statistically lower in the MS cohort compared to the NMO cohort (0.73 vs 1.68, p=0.018). AGS-NMO scores were statistically lower in the HC cohort compared to the NMO cohort (0.68 vs 1.68, p=0.021).

AGS-NMO Using VH1 Genes Only.

This analysis resulted in 6 codons from VH1 antibody genes that had accumulated mutations to a statistically higher magnitude in NMO antibody databases compared to MS or HC antibody databases. The VH1 codons are: 47; 54; 70; 79; 84; 91. The average AGS-NMO score for the NMO cohort is 1.319 (range 0.57 to 2.17). The average AGS-NMO score for the MS cohort is 0.369 (range 0.09 to 0.71). The average AGS-NMO score for the HC cohort is 0.473 (range 0.00 to 0.67). As expected, AGS-NMO scores were statistically lower in the MS cohort compared to the NMO cohort (0.37 vs 1.32, p=0.006). AGS-NMO scores were statistically lower in the HC cohort compared to the NMO cohort (0.47 vs 1.32, p=0.006).

AGS-NMO Using VH1 and VH4 Genes in Combination.

When the AGS scores are calculated from a combination of the VH1 and VH4 codons, the average AGS-NMO score for the NMO cohort is 2.786 (range 1.75 to 4.17). The average AGS-NMO score for the MS cohort is 1.009 (range 0.16 to 1.83). The average AGS-NMO score for the HC cohort is 0.873 (range 0.00 to 1.73). As expected, AGS-NMO scores were statistically lower in the MS cohort compared to the NMO cohort (1.01 vs 2.79, p=0.0006). AGS-NMO scores were statistically lower in the HC cohort compared to the NMO cohort (0.87 vs 2.79, p=0.0005).

### Example 3

### Discussion

In summary, the inventor has used the same approach that led to the discovery of the AGS for MS to identify an AGS that distinguishes NMO from MS, HC and other neurological diseases. A few modifications in our approach have been made to further refine the AGS for NMO, including the pre-filtering of the data through a “replacement only” screen as described above and normalization of the score to take into consideration the number of sequences in each sample's antibody repertoire.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. Certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### V. REFERENCES

The following references, to the extent that they provide exemplary procedural or other details supplementary to those set forth herein, are specifically incorporated herein by reference:
U.S. Pat. No. 4,582,788
U.S. Pat. No. 4,656,127
U.S. Pat. No. 4,659,774
U.S. Pat. No. 4,682,195
U.S. Pat. No. 4,683,194
U.S. Pat. No. 4,683,195
U.S. Pat. No. 4,683,202
U.S. Pat. No. 4,800,159
U.S. Pat. No. 4,816,571
U.S. Pat. No. 4,883,750
U.S. Pat. No. 4,946,773
U.S. Pat. No. 4,959,463
U.S. Pat. No. 4,965,188
U.S. Pat. No. 5,130,238
U.S. Pat. No. 5,141,813
U.S. Pat. No. 5,169,766
U.S. Pat. No. 5,264,566
U.S. Pat. No. 5,279,721
U.S. Pat. No. 5,428,148
U.S. Pat. No. 5,554,744
U.S. Pat. No. 5,574,146
U.S. Pat. No. 5,602,244
U.S. Pat. No. 5,605,798
U.S. Pat. No. 5,645,897
U.S. Pat. No. 5,705,629
U.S. Pat. No. 5,840,873
U.S. Pat. No. 5,843,640
U.S. Pat. No. 5,843,650
U.S. Pat. No. 5,843,651
U.S. Pat. No. 5,843,663
U.S. Pat. No. 5,846,708
U.S. Pat. No. 5,846,709
U.S. Pat. No. 5,846,717
U.S. Pat. No. 5,846,726
U.S. Pat. No. 5,846,729
U.S. Pat. No. 5,846,783
U.S. Pat. No. 5,849,481
U.S. Pat. No. 5,849,483
U.S. Pat. No. 5,849,486
U.S. Pat. No. 5,849,487
U.S. Pat. No. 5,849,497
U.S. Pat. No. 5,849,546
U.S. Pat. No. 5,849,547
U.S. Pat. No. 5,851,770
U.S. Pat. No. 5,851,772
U.S. Pat. No. 5,853,990
U.S. Pat. No. 5,853,992
U.S. Pat. No. 5,853,993
U.S. Pat. No. 5,856,092
U.S. Pat. No. 5,858,652
U.S. Pat. No. 5,861,244
U.S. Pat. No. 5,863,732
U.S. Pat. No. 5,863,753
U.S. Pat. No. 5,866,331
U.S. Pat. No. 5,866,337
U.S. Pat. No. 5,866,366
U.S. Pat. No. 5,900,481
U.S. Pat. No. 5,905,024
U.S. Pat. No. 5,910,407
U.S. Pat. No. 5,912,124
U.S. Pat. No. 5,912,145
U.S. Pat. No. 5,912,148
U.S. Pat. No. 5,916,776
U.S. Pat. No. 5,916,779
U.S. Pat. No. 5,919,626
U.S. Pat. No. 5,919,630
U.S. Pat. No. 5,922,574
U.S. Pat. No. 5,925,517
U.S. Pat. No. 5,925,525
U.S. Pat. No. 5,928,862
U.S. Pat. No. 5,928,869
U.S. Pat. No. 5,928,870
U.S. Pat. No. 5,928,905
U.S. Pat. No. 5,928,906
U.S. Pat. No. 5,929,227
U.S. Pat. No. 5,932,413
U.S. Pat. No. 5,932,451
U.S. Pat. No. 5,935,791
U.S. Pat. No. 5,935,825
U.S. Pat. No. 5,939,291
U.S. Pat. No. 5,942,391
U.S. Pat. No. 5,952,174
Ausubel et al., Current Protocols in Molecular Biology, John Wiley & Sons, NY, 1989.
Barkhof et al., Brain, 120(Pt11):2059-2069, 1997.
Barany, et al., Proc. Natl. Acad. Sci. USA, 88:189-193, 1991.
Baranzini et al., J. Immunol., 163:5133-5144, 1999.
Beck et al., Arch. Ophthalmol., 121:944-949, 2003.
Bellus, J. Macromol. Sci. Pure Appl. Chem., A31(1): 1355-1376, 1994.
Bennett et al., J. Neuroimmunol., 199:126-132, 2008.
Both et al., Mol. Cell Biol., 10:5187-5196, 1990.
Berx et al., N. Engl. J. Med., 346:158-164, 2002.
Brezinschek et al., J. Clin. Invest., 99:2488-2501, 1997.
Brezinschek et al., J. Immunol., 155:190-202, 1995.
Brezinschek et al., J. Immunol., 160:4762-4767, 1998.
Buluwela and Rabbitts, Eur. J. Immunol., 18:1843-1845, 1988.
Cepok et al., Brain, 124:2169-2176, 2001.
Cepok et al., Brain, 128:1667-1676, 2005.
Chothia and Lesk, J. Mol. Biol., 196:901-917, 1987.
Chothia et al., J. Mol. Biol., 227:799-817, 1992.
Cole et al., Neurology, 51:885-887, 1998.
Colombo et al., J. Immunol., 164:2782-2789, 2000.
Cook and Tomlinson, Immunol. Today, 16:237-242, 1995.
Corcione et al., Proc. Natl. Acad. Sci. USA, 101:11064-11069, 2004.
Dalton et al., Ann. Neurol., 52:47-53, 2002.
Damle et al., Blood, 94:1840-1847, 1999.
de Arruda et al., Expert. Rev. Mol. Diagn., 2:487-496, 2002.
Domiati-Saad and Lipsky, J. Immunol., 161:1257-1266, 1998.
Dorner et al., J. Immunol., 158:2779-2789, 1997.
Dorner et al., Eur. J. Immunol., 28:657-668, 1998a.
Dorner et al., J. Immunol., 160:2831-2841, 1998b.
Dorner et al., Eur. J. Immunol., 28:3384-3396, 1998c.
Dorner et al., J. Immunol., 163:1027-1036, 1999.
EP 201,184
EP 237,362
EP 258,017
EP 266,032
EP 320 308
EP 329,822
EP 50,424
EP 84,796
Esiri, Lancet., 2:478, 1977.
Farner et al., J. Immunol., 162:2137-2145, 1999.
Foster et al., Eur. J. Immunol., 29:3122-3132, 1999.
Freedman et al., Arch. Neurol., 62:865-870, 2005.
French Patent 2,650,840
Froehler et al., Nucleic Acids Res., 14(13):5399-5407, 1986.
Frohman, In: PCR Protocols: A Guide To Methods And Applications, Academic Press, N.Y., 1990.
Frohman et al., Neurology, 61:602-611, 2003.
Genain et aL, Nat. Med., 5:170-175, 1999.
Great Britain Appln. 2 202 328
Guddat et al., Proc. Natl. Acad. Sci. USA, 90:4271-4275, 1993.
Hamblin et al., Blood, 94:1848-1854, 1999.
Hansen et al., Int. Arch. Allergy Immunol., 123:36-45, 2000.
Hansen et al., Scand. J. Immunol., 57:470-479, 2003.
Harp et al., J. Neuroimmunol., 183:189-199, 2007.
Hauser et al., N. Engl. J. Med., 358:676-688, 2008.
Hayashi et al., Int. J. Mol. Med., 20:247-253, 2007.
Huang et al., J. Clin. Invest., 89:1331-1343, 1992.
Huang et aL, Clin. Exp. Immunol., 112:516-527, 1998.
Humphries et al., In: Molecular Diagnosis of Genetic Diseases, Elles (Ed.), 321-340, 1996.
Humphries et aL, Nature, 331:446-449, 1988.
Innis et al., Proc. Natl. Acad. Sci. USA, 85(24):9436-9440, 1988.
Jones, Nature, 199:280-282, 1963.
Kabat et al., Am. J. Med. Sci., 219:55-64, 1950.
Kabat et al., Am. J. Med., 4:653-662, 1948.
Kabat et al., In: Sequences of Proteins of Immunological Interest, United States Department of Health and Human Services, Washington, D.C., 1983.
Kabat et al., In: Sequences of Proteins of Immunological Interest, 4^{th} Ed., United States Department of Health and Human Services, Washington, D.C., 1987.
Kirkham and Schroeder, Semin. Immunol., 6:347-360, 1994.
Kodaira et al., J. Mol. Biol., 190:529-541, 1986.
Koelsch et al., J. Clin. Invest., 117(6):1558-65, 2007.
Komher, et al., Nucl. Acids. Res. 17:7779-7784, 1989.
Korteweg et al., Lancet. Neurol., 5:221-227, 2006.
Kraj et al., J. Immunol., 158:5842-5832, 1997.
Kuppuswamy et al., Proc. Natl. Acad. Sci. USA, 88:1143-1147,1991.
Kwoh et al., Proc. Natl. Acad. Sci. USA, 86:1173, 1989.
Kwok and Chen, Curr Issues Mol. Biol., 5(2):43-60, 2003.
Kwok, Annu. Rev. Genomics Hum. Genet., 2:235-258, 2001.
Lambracht-Washington et al., J. Neuroimmunol., 186(1-2):164-76, 2007.
Landegren et al., Science 241:1077-1080, 1988.
Lee et al., J. Mol. Biol., 195:761-768, 1987.
Lu et al., Biopolymers, 73:606-613, 2004.
Magliozzi et al., J. Neuroimmunol., 148:11-23, 2004.
Martin-Mdel and Monson, Front Biosci., 12:2735-2749, 2007.
Maxam et al., Proc. Natl. Acad. Sci. USA, 74:560, 1977.
McDonald et al., Ann. Neurol., 50:121-127, 2001.
Meffre et al., Nat. Immunol., 1:207-213, 2000.
Meyers et al., Science, 230:1242, 1985.
Mockridge et al., Autoimmunity, 37:9-15, 2004.
Modrich, Ann. Rev. Genet., 25:229-253, 1991.
Monson et al., Eur. J. Immunol., 30:1597-1605, 2000.
Monson et al., J. Neuroimmunol., 158:170-181, 2005.
Mullis et al., Cold Spring Harbor Symp. Quant. Biol. 51:263-273, 1986.
Nickerson et al., Proc. Natl. Acad. Sci. USA, 87:8923-8927,1990.
Nyren et al., Anal. Biochem. 208:171-175, 1993.
Ohara et al., Proc. Natl. Acad. Sci. USA, 86:5673-5677, 1989.
Oppezzo et al., Eur. J. Immunol., 34:1423-1432, 2004.
Orita et al., Genomics, 5:874-879, 1989.
O'Riordan et al., Brain, 121(Pt3):495-503, 1998
Owens et al., Ann. Neurol., 43:236-243, 1998.
Owens et al., J. Immunol., 171:2725-2733, 2003.
Owens et al., J. Immunol., 179:6343-6351, 2007.
Ozawa et al., Brain, 117(Pt 6):1311-1322, 1994.
Paolino et al., J. Neurol. Neurosurg. Psychiatry, 60:572-575, 1996.
Pascual and Capra, Arthritis Rheum., 35:11-18, 1992.
PCT Appln. PCTUS8700880
PCT Appln. PCTUS8901025
PCT Appln. WO 88/0315
PCT Appln. WO 89/06700
PCT Appln. WO 89/06700
PCT Appln. WO 9001069
PCT Appln. WO 9322456
PCT Appln. WO 95/1995
PCT Appln. WO 9102087
PCT Appln. WO 92/5712
Polman et al., Ann. Neurol., 58:840-846, 2005.
Prezant et al., Hum. Mutat., 1:159-164, 1992.
Pugh-Bernard et al., J. Clin. Invest., 108:1061-1070, 2001.
Qin et al., J Clin Invest 1998. 102: 1045-1050, 1998.
Raine et al., Ann. Neurol., 46:144-160, 1999.
Reimer & Jensen-Jarolim, Immunol Lett. 112(1):1-5 (2007).
Ritchie et al., J. Immunol., 173:649-656, 2004.
Rogozin and Diaz, J. Immunol., 172: 3382-3384, 2004.
Ruano et al., Nucl. Acids Res., 19:6877-6882, 1991.
Ruano et al., Nucl. Acids Res., 17:8392, 1989.
Sambrook et al., In: Molecular cloning, Cold Spring Harbor Laboratory Press, Cold Spring Harbor, NY, 2001.
Sanger et al., J. Molec. Biol., 94:441, 1975.
Sayle and Milner-White, Trends Biochem. Sci., 20:374, 1995.
Serafini et al., Brain Pathol., 14:164-174, 2004.
Sheffield et al., Proc. Natl. Acad. Sci. USA, 86:232-236, 1989.
Shen et al., Proc. Natl. Acad. Sci. USA, 84:8563-8567, 1987.
Shlomchik et al., Nature, 328:805-811, 1987.
Siritho and Freedman, J. Neurol. Sci., 2009 (In Print)
Soderstrom et al., Neurology, 50:708-714, 1998.
Sokolov, Nucl. Acids Res. 18:3671, 1990.
Souza et al., J. Immunol., 179:3153-3160, 2007.
Stevens et al., Biotechniques, 34:198-203, 2003.
Storch and Lassmann, Curr. Opin. Neurol., 10:186-192, 1997.
Stuve et al., Drugs, 68:73-83, 2008.
Stuve et al., Ann. Neurol., 59:743-747, 2006
Syvanen et al., Genomics 8:684-692, 1990.
Tanaka and Nei, Mol. Biol. Evol., 6:447-459, 1989.
Tian et al., Mol. Immunol., 44:2173-2183, 2007.
Tintore et al., Am. J. Neuroradiol., 21:702-706, 2000.
Turki et al., J. Clin. Invest., 95:1635-1641, 1995.
Uccelli et al., Trends Immuno., 26:254-259, 2005.
Ugozzoll et al., GATA 9:107-112, 1992.
Vargas-Madrazo et al., J. Mol. Evol., 1994.38:100-104, 1994.
Voswinkel et al., Ann. NY Acad. Sci., 815:312-315, 1997.
Walker et al., Proc. Natl. Acad. Sci. USA, 89:392-396, 1992.
Wardemann et al., Science, 301:1374-1377, 2003.
Wartell et al., Nucl. Acids Res., 18:2699-2706, 1990.
Winges et al., J. Neuroimmunol., 192(1-2):226-234. 2007.
Winter et al., Proc. Nat. Acad. Sci. USA, 82:7575, 1985.
Yurasov et al., J. Exp. Med., 201:703-711, 2005.
Zheng et al., J. Cin. Invest., 113:1188-1201, 2004.

What is claimed is:

1. A method for selecting a human subject having or at risk of developing neuromyelitis optica (NMO) comprising:
(a) providing a B-cell containing sample from a subject, or DNA or RNA isolated therefrom;
(b) determining the VH1 and/or VH4 structure of VH1/VH4-expressing B-cells from said subject,
(c) determining the mutational frequency VH1 and/or VH4 genes;
(d) identifying the presence or absence of a codon signature associated with NMO or risk of NMO; and
(e) selecting patients exhibiting said codon signature.

2. The method of claim 1, wherein said NMO codon signature comprises a mutation in VH1 at codon 47, 54, 70, 79, 84 and/or 91.

3. The method of claim 2, wherein said codon signature comprises mutations at 2, 3, 4, 5 or all 6 of said codons.

4. The method of claim 1, wherein said NMO codon signature comprises a mutation in VH4 at codon 36, 39, 45, 46, 50, 59, 61, 65, 67, 70, 86 and/or 90.

5. The method of claim 4, wherein said codon signature comprises mutations at 2, 3, 4, 5, 6, 7, 8, 9, 10, 11 or all 12 of said codons.

6. The method of claim 1, wherein said NMO codon signature comprises a mutation in codons 47, 54, 70, 79, 84 and/or 91 of VH1 and codons 36, 39, 45, 46, 50, 59, 61, 65, 67, 70, 86 and/or 90 of VH4.

7. The method of claim 6, wherein said codon signature comprises mutations at 2, 3, 4, 5, 6, 7, 8, 9, 10, 11, 12, 13, 14, 15, 16, 17 or all 18 of said codons.

8. The method of claim 1, further comprising assessing one or more traditional NMO risk factors.

9. The method claim 1, wherein assessing comprises sequencing.

10. The method of claim 1, wherein assessing comprises PCR.

11. The method of claim 1, wherein said B-cell(s) isare obtained from cerebrospinal fluid (CSF).

12. The method of claim 11, further comprising assessing J chain usage, J chain length and/or CDR3 length.

13. The method of claim 1, wherein said B-cell(s) isare obtained from peripheral blood.

14. The method of claim 13, further comprising assessing J chain usage, J chain length and/or CDR3 length.

15. The method of claim 1, further comprising making a treatment decision based on the presence of said codon signature.

16. A method of screening for an agent useful in treating neuromyelitis optica (NMO) comprising:
(a) providing an antibody produced by a VH1 or VH4-expressing B-cell, said antibody comprising mutations at two or more codons selected from the group consisting of codons 47, 54, 70, 79, 84 of VH1 or codons 36, 39, 45, 46, 50, 59, 61, 65, 67, 70, 86 and 90 of VH4;
(b) contacting said antibody with a candidate ligand; and
(c) assessing binding of said candidate ligand to said antibody,
wherein binding of said candidate ligand to said antibody identifies said candidate ligand as useful in treating NMO.

17. The method of claim 16, wherein said candidate ligand is a peptide or a peptoid.

18. The method of claim 16, wherein a NMO codon signature comprises at least one mutation in both VH1 and VH4 antibodies.

19. The method of claim 16, wherein a NMO codon signature comprises mutations at 2, 3, 4, 5, 6, 7, 8, 9, 10, 11, 12, 13, 14, 15, 16, 17 or all 18 of said codons.

20. A method of treating a subject having or at risk of developing neuromyelitis optica (NMO) comprising administering to said subject a ligand that binds to a VH1 or VH4 antibody comprising mutations at two or more codons selected from the group consisting of codons 47, 54, 70, 79, 84 and 91 of VH1 or codons 36, 39, 45, 46, 50, 59, 61, 65, 67, 70, 86 and 90 of VH4.

21. The method of claim 20, wherein said ligand is a peptide or a peptoid.

22. The method of claim 20, wherein a NMO codon signature comprises at least one mutation in both VH1 and VH4.

23. The method of claim 20, wherein a NMO codon signature comprises mutations at 2, 3, 4, 5, 6, 7, 8, 9, 10, 11, 12, 13, 14, 15, 16, 17 or all 18 of said codons.

24. The method of claim 20, wherein said ligand is linked to a toxin or B-cell antagonist.

25. The method of claim 18 or 22, comprises multiple mutations in both VH1 and VH4 antibodies.
